(19) United States
(12) Patent Application Publication
(10) Pub. No.: US 2003/0163353 A1
(43) Pub. Date: Aug. 28, 2003

(54) Method and system for patient preference determination for treatment options

(76) Inventors: Bryan Luce, Bethesda, MD (US); Dennis Revicki, Ellicott City, MD (US)

Correspondence Address: Supervisor, Patent Prosecution Services, PIPER RUDNICK LLP, 1200 Nineteenth Street, N.W., Washington, DC, 20036-2412, US

(21) Appl. No.: 10/351,535

(22) Filed: Jan. 27, 2003

Related U.S. Application Data
(60) Provisional application No. 60/350,939, filed on Jan. 25, 2002.

(51) Int. Cl. G06F 17/60; A61B 5/00

(52) U.S. Cl. 705/2; 600/300

(57) ABSTRACT

A method and system for determining treatment preference information. Individuals are queried for data such as demographic information, preference factors, and tradeoff selections. The preference factors include, for example, relief from symptoms, cost of treatment, side effects of treatment, frequency of treatment required, and mode of administration of treatment. Using repositories of treatment related information, the preference factors are evaluated with regard to treatment options, then treatment preferences are refined via selection of tradeoff preferences. Treatment options are then ranked or otherwise compared, based on analyzed preference information.

This application claims priority from U.S. Provisional Patent Application Ser. No. 60/350,939 filed Jan. 25, 2002. The entirety of that provisional application is incorporated herein by reference.

### BACKGROUND OF THE INVENTION

1. Field of the Invention

The present invention relates to a method and system for matching patient preference data with products, such as drugs and other pharmaceuticals, and in particular to a computerized method and system for receiving preference data on patients and using this data with a therapeutic product and other treatment-related information to determine a treatment preference for each patient.

2. Background of the Technology

In general, two types of problems exist with regard to patient preferences and products for treatment, such as medications.

One type of problem is a clinical problem, in that individual patients have preference profiles—each patient has a set of personal preferences unique to that individual. For example, if a patient needs a hypertensive drug, a number of choices are available. However, every hypertensive drug has a different profile, based on such features as cost, side effects, relief from symptoms, frequency of treatment required, and mode of administration of treatment. As to cost, the patient may have more out-of-pocket cost for a newer drug, for example, while for an older drug, the patient may pay very little.

An illustrative example of drug selection for high blood pressure will now be presented. Some hypertensive drugs produce the side effect of anxiety; others produce sleeplessness; others upset the stomach; and others depress the libido; etc. In the existing art, a treatment specialist, such as a doctor, examines the patient from a clinical perspective and evaluates treatment primarily focusing on reducing the blood pressure. The treatment specialist then typically selects the drug that is most appropriate for bringing down the patient's blood pressure.

But this specialist may not really know the profile of the set of available drugs, and very likely does not know the patient's personal preference profile. One problem is that, when a drug is prescribed for a patient, it is typically prescribed without information about that patient relative to known drug opportunities. As a result, there may be a mismatch with patient preference, which can lead to many other problems, including low satisfaction with treatment and/or lack of compliance with drug dosage requirements. For example, with existing treatment methods, patients disliking a drug prescribed may simply underdose themselves (a potential negative health effect) or stop therapy altogether, or additional costs may ultimately be incurred because the patient returns to the doctor to try alternatives.

Additional, cascading types of problems with the prior art can also occur as a result of the clinical effect of failure to incorporate patient preference in the treatment selection process. For example, the patient may initially be dissatisfied because the doctor does not ask the right questions about the patient's lifestyle and preferences with regard to such issues as side effects. Patient dissatisfaction can lead not only to failure to diligently comply with treatment programs or increased costs, due to return visits, but also can result in overall dissatisfaction with health care providers. In actuality, another available drug for the patient's symptoms may make that patient feel better in some ways. The alternate drug may also make the patient feel worse in other ways or have other negative features. However, the patient may prefer the alternate drug overall, or be willing to otherwise overcome certain negative features, such as a higher price.

A second type of problem relating to patient preferences and products is associated with the product providers, such as drug companies. Drug companies are continuously in the process of developing and promoting drugs for those people who are best suited for them. One problem for drug companies is a lack of information usable for such purposes as marketing, research, and development that is directed to individual patient preferences. While companies are able to determine through clinical trials such information as side effects and other relevant information, this information does not tell these companies which features patients typically prefer when faced with tradeoffs or other comparative aspects of multiple products for the same therapeutic category.

Thus there remains an unmet need for a method and system to address the problem of failure to target the right drug or other treatment to an individual, based on that individual's preference profile, and the problem of inability of drug companies and other treatment product providers to target resources appropriately for such purposes as marketing, research and development. There also remains an unmet need for methods and systems that assist with minimizing unsatisfactory care and associated potentially increased costs.

### SUMMARY OF THE INVENTION

The present invention provides a method and system for determining treatment preference information. In one embodiment, individuals are queried for such data as demographic information, preference factors, and tradeoff selections. The preference factors include, for example, relief from symptoms, cost of treatment, side effects of treatment, frequency of treatment required, and mode of administration of treatment. In one embodiment, input patient preference data are used with repositories (e.g., databases) of treatment related information, and then treatment preferences are refined via use of selectable options for tradeoff preferences. In one embodiment, treatment options are then ranked or otherwise compared.

In particular, in one embodiment, the present invention links the profiles of treatments, such as medications, currently on the marketplace, as well as those in development, so long as therapeutic profile information is available, to individual preferences of individual patients in a variety of domains. One embodiment uses a processor contained within or coupled to a data collection vehicle (also referred to interchangeably herein as a “terminal”), such as a hand-held personal digital assistant (PDA), a laptop computer, or other stand-alone or networked devices, such as a network-connected personal computer (PC) that presents the user with a series of queries that ascertains preferences. In one embodiment, the responses are used in conjunction with an algorithm within a software program to link the patient preferences to medicine or other treatment profiles, and the results are communicated to, for example, the patient or the treating physician. The findings can thus be used in the prescription decision-making or other treatment process.

In another embodiment, the patient preference information is collected, and, for example, categorized and used to prepare reports on the findings. The data are thus usable for such purposes as to drive market share based decisions, to provide a clinical decision-making tool from, for example, the perspective of a managed health care organization, or to generate patient level market research data to support research and development. For example, such data are becoming more important as pharmaceutical manufacturers increasingly directly target consumers in advertising and other promotional and marketing efforts.

One embodiment of the present invention begins with an introductory page that collects basic demographic information, such as age, sex, and geographic location of residence. This embodiment also includes two user selection sections, for preference ranking and tradeoffs, which allow the user to consider different domains and issues and to rank these issues according to importance to the patient. Example domains and issues for preference ranking include relief from symptoms, cost of treatment, side effects of treatment, frequency of treatment required, and mode of administration of treatment. In one embodiment, these items are ranked and numerically scored, such as by use of a point range corresponding to a selection varying between “not important” to “very important.”

A tradeoff section allows the user to make tradeoffs, so that, for example, the user may select between treatment characteristics that reflect closely ranked preferences for the user. For example, the user may select between medication A, which provides complete relief 85% of time and costs $15.00 per month, and medication B which provides complete relief 70% of the time and cost $5.00 per month. In one embodiment, the degree of preference for the user, with an associated score, can also be inputed.

In one embodiment, the patient preference and tradeoff information, in conjunction with other data, such as data on products and costs, is then used to produce a ranking of appropriate products. In one embodiment, determination, selection, and evaluation of preference occurs using adaptive conjoint analysis or discrete choice model analysis, as is known in the art.

In one embodiment, the inputed patient preference information is collected in a preference repository, such as a database. Other repositories of this embodiment of the present invention include a repository of therapeutic product information and a repository of cost-related information. In one embodiment, the product information is collected and input into a repository to form product profiles. An embodiment of the present invention further provides capability for industry users to access and input information for new products, such as drugs, into existing categories of similar products.

In an embodiment of the present invention, reports are generated on a regular basis (e.g., monthly). In one embodiment, syndicated reports are also generated, for example, on the basis of therapeutic categories, and are tailorable to particular industry or client needs.

Additional advantages and novel features of the invention will be set forth in part in the description that follows, and in part will become more apparent to those skilled in the art upon examination of the following or upon learning by practice of the invention.

### BRIEF DESCRIPTION OF THE FIGURES

FIG. 1 presents various components of a standalone system for evaluating patient preference information, in accordance with an embodiment of the present invention;

FIG. 2 shows the components of a network-based system for evaluating patient preference information, in accordance with an embodiment of the present invention;

FIG. 3 is a flow chart of an overview of the data gathering and analysis method for individual patient medication preference applications, in accordance with an embodiment of the present invention;

FIG. 4 is a flow chart of functions involved in an exemplary method for receiving and evaluating patient preference information, in accordance with an embodiment of the present invention; and

FIGS. 5-14 present exemplary graphical user interface (GUI) screens for patient preference data input, in accordance with one embodiment of the present invention.

### DETAILED DESCRIPTION

An embodiment of the present invention, centers on linking patient preferences to treatment (e.g., medicine) profiles in order to assist the prescribing process and to provide data for other purposes, such as market research. The present invention thus provides a system and method for increasing compliance, improving outcomes, and lowering costs.

Importantly, individual patients inherently have individual preference profiles. At least within the United States market, patient preferences are likely to play an increasingly key role in patient demand and selection for products, especially those products that enhance quality of life. This is true partly because of direct-to-consumer (DTC) promotion by pharmaceutical companies, but perhaps more significantly because of increased health consumer empowerment via evidence of patient benefit available on networks, such as the Internet, and elsewhere. As well, it is important to note that out of pocket expenses for the newer products will likely become more of an issue in the future. Patient preference plays a key role here as well.

One embodiment of the present invention provides the capability to test patient-specific market acceptance of selected products relative to direct competitors for these products (e.g., products presently on the market and other products in development) and, importantly, this can be performed outside the clinical trial program. In fact, the product itself is not needed, just its therapeutic profile, based on the clinical studies and expectations for the product. The present invention provides the capability to answer key market-oriented questions such as:

1. What characteristics of the drug appeal most to individual patients relative to alternative options?

2. What is the required relative strength of the various side effects or Quality of Life (QOL)-enhancing effects that best predict patient switching patterns?

3. What patient preference profiles are most compatible with the preference profile of the drug at issue?

In operation, an embodiment of the present invention includes a method and system, such as a computer-based method and system for operation on a network, such as the Internet, that allows matching of patient preferences with specific drugs or other pharmaceutical products. Functionality of the system is based on a series of algorithms that link the profiles of drugs or other pharmaceuticals to specific patient preferences. In one embodiment, patent preferences are categorized into different domains.

An embodiment of the present invention further provides capability for industry users to access and input information for new products, such as drugs, into existing categories of similar products. In an embodiment of the present invention, reports are generated on a regular basis (e.g., monthly). In one embodiment, syndicated reports are also generatable, for example, on the basis of therapeutic categories, and are tailorable to particular industry or client needs.

An embodiment of the present invention includes three repositories (e.g., databases) that interface to support determination of patient preference information. The first repository is a treatment database, (e.g., containing medication information) (also referred to interchangeably herein as a “repository of treatment option information”). The second repository is a health care cost database containing health care financial information and other information allowing, for example, determination of patient-specific managed care plan information for particular medications (also referred to interchangeably herein as a “repository of medication costs information”). The third repository contains patient-specific preference and preference-related information (also referred to interchangeably herein as a “repository of treatment preference information”).

The treatment repository of one embodiment of the present invention includes information on therapeutic category (e.g., depression, allergies, hypertension) using clinical data (e.g., data that is submitted to the Food and Drug Administration (FDA); also other available data known about a medication) that is vetted to ensure that the data is valid or well recognized by the clinical community. This data is based, for example, on clinical trials and collation of relevant information for a number of predetermined dimensions, such as dosage (e.g., taken two times per day) and side effects (e.g., nausea, anxiety, sleeplessness, sexual functioning, pain), along with some weighting of the intensity of these effects (e.g., severe nausea; slight pain), as well as other factors, such as frequency of incidence, likelihood, probability, and mode of administration (e.g., oral, intramuscular injection). In an embodiment of the present invention, this information is reviewed and normalized, for example, by experts in the field. All of this therapeutic information is maintained in a continuously updated repository. The repository is maintained for all clinical categories for which there are multiple medication options.

Another aspect of the process of the present invention involves collection of cost information, and in particular, managed care and other health care financial information in a repository. This information is included in the database to allow it to be used in tradeoffs involving cost issues, including actual out-of-pocket costs for individuals in managed health care plans. Information gathered includes scope of coverage of the health plan, such as, but not limited to, medications for which the individual pays a high co-pay, and those for which the individual has a low co-pay.

In operation, each patient or other user inputs information on preferences into an interactive system. For example, in one embodiment, a query and response system is provided (e.g., via a terminal, such as a PC, mini-computer, microcomputer, mainframe computer, telephone, hand-held device (e.g., PDA), or other device with capability for input and a processor or coupling to a processor), which is tailored for each therapeutic category. In this embodiment, the user inputs demographic information in response to a request for this information. Then the user indicates or ranks the importance of various Preference Factors that affect treatment selection. In one embodiment, the Preference Factors include relief from symptoms, cost of treatment, side effects from treatment, frequency of treatment, and mode of administration. For example, in one embodiment, the user selects a preferred point for each Preference Factor on a variable scale ranging from “not important” to “very important.”

In one embodiment, following analysis of the various Preference Factors for the therapeutic category and available treatments (e.g., medications), as necessary, a series of tradeoff queries are generated (this overall process is also interchangeably referred to herein as the “analysis and comparison process”). For example, the user may be asked to select on a ranging scale (e.g., ranging from “absolutely prefer” to “hardly prefer”) between a preference for a first medication that has certain side effects and a second medication that has differing side effects. A variable number of tradeoff queries are generated, depending on the user's preference factors and the information contained in the treatment database relating to the therapeutic category.

For example, in one embodiment, the preference factors are compared to corresponding information in the treatment repository, and if the preference factors differ by less than a predetermined value (e.g., 30%), tradeoffs are generated, while if the values differ by greater than the predetermined value, the user's preference is assumed based on this difference. This example is merely illustrative, as many techniques are useable for weighing and comparing preference information, in accordance with the present invention. See, e.g., Mandy Ryan, Ph.D., “Using Consumer Preferences in Health Care Decision Making: The Application of Conjoint Analysis,” The Office of Health Economics, London, England (1996) (discussing conjoint analysis); Vic Adamowicz and Reed Johnson, “Stated Preference Methods in Health Economics,” presentation at iHEA Annual Meeting, York, UK (July 2001); and Johnson F R., Banzhaf M S, Desvouges W H, “Willingness to Pay for Improved Respiratory and Cardiovascular Health. A Multiple-format, Stated-preference Approach,” Health Economics (2000) (discussing comparison of health choices), each of which is hereby incorporated by reference.

The following is one example application of the analysis and comparison process, which is provided for illustrative purposes only. In this example, the user selects preference information using a scale from 0 to 100 (corresponding to a range from “not important” at 0 to “very important” at 100). These received preference factors are used as follows:

1. The user selects a percentage of importance for each Preference Factor.

2. The percentages are compared and entered for each Preference Factor with the corresponding percentages determined for each of the other Preference Factors. With four Preference Factors for example, there are six pairs of comparisons among the Factors (i.e., first and second Factors, first and third Factors, first and fourth Factors, second and third Factors, second and fourth Factors, and third and fourth Factors). If the difference in the percentages between the two Factors in a combination equals or is greater than 30, a score of five is assigned to the Preference Factor with the higher percentage in the pair.

3. For those combinations with a difference less than 30, a series of trade-offs is determined and presented for the user to rank. For example, in the case of three medications being evaluated, each medication may have a characteristic relating to each Preference Factor (e.g., for relief from symptoms, medication #1 has the response of “Complete relief for 85% of time” and medication #2 has the response “Complete relief for 50% of time”), which is stored, for example, in the repository of treatment option information. From a pre-defined table or other database source or selection process, two state pairs are defined for each Preference Factor combination. Each state combines two treatment responses relevant to the Preference Factor for the pair of medications being compared. In one embodiment, each state presents different responses for each Preference Factor of interest. The pair of responses for each state may, for example, be for the same medication or for different medications.

In this example, trade-offs are determined as follows:

a. For the Preference Factor combination with a difference of less than 30, greater detail must be received from the user in order to provide a score. Each state pair as created above is presented to the user, and the user selects the preferable state and scores it on, for, example, a scale, such as a scale from 1 to 5 (ranging from “hardly prefer” to “most prefer”).

b. For the preferred state, the selected score is added to the total score for each response. As indicated above, each response is medication-specific.

4. The results for each treatment being evaluated are calculated:

a. The scores for each response are summed;

b. The sum for each response is divided by the total number of responses with scores;

c. Each response sum of (b) is divided by the sum of (a) to produce a weighted average for each response;

d. The weighted averages from (c) are divided by the ranking of each Preference Factor. For example, this ranking may be pre-defined but could also, for example, be calculated based on predetermined factors (e.g., relative severity); and

e. The weighted medication scores from (d) are summed and divided by the number of medications to provide the ranking for each medication.

The rankings for each medication and the associated responses for each Preference Factor are then collected and optionally presented to the user or, for example, to a physician or drug company.

In embodiments of the present invention, the user's input of preference factors and other information, such as demographic information and tradeoff selections, occurs on a terminal at a treatment location, such as a doctor's office.

In one embodiment, the terminal is coupled to a network, such as the Internet, and the repositories are located remotely from the terminal, such as on a server on the network. In this embodiment, the user inputs preference information, for example, while in the waiting room prior to a doctor's visit, or during or after the visit.

In another embodiment, the user simply inputs information on a terminal on the network while at any location (e.g., while at home by accessing a server via the Internet). In yet another embodiment, selected treatment specialists (e.g., allergists) are recruited to input data regarding specific therapeutic categories for patients in those categories, or for groups of patients interested in screening, for example, in particular therapeutic categories (e.g., managed health care group participants interested in high blood pressure screening and treatment).

Regardless of how input, in one embodiment, the user input information is then collected in the patient preference repository. In another embodiment, the patient preference repository is accessible and usable for a variety of other purposes and usable in a correlated or integrated fashion with the treatment repository. For example, the information in these repositories may be analyzed and/or accessed by drug manufacturers for use in marketing and research and development decisions (e.g., high preference trend for certain medicines or for certain Preference Factors (e.g., symptom relief) by patients in the therapeutic category), or by health care management organizations when ensuring preferred medications are available for patient insurance programs (e.g., ensure patient satisfaction and compliance; make evaluations of program preferred medications, such as where no clear trend in preferences and one medication is significantly less expensive).

References will now be made in detail to embodiments of the present invention, examples of which are illustrated in the accompanying drawings.

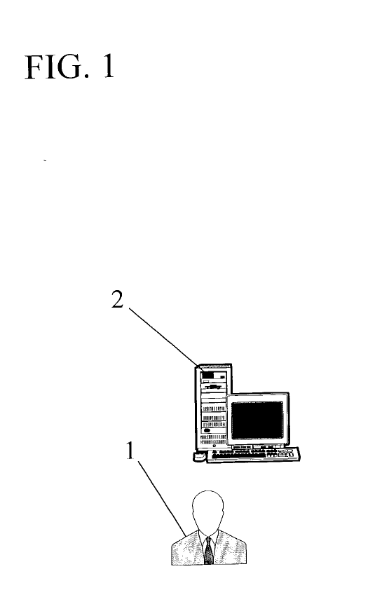

As shown in FIG. 1, in an embodiment of the present invention, data for use in the system is collected from a user 1 via a terminal 2, such as a PC, minicomputer, mainframe computer, microcomputer, telephonic device, or wireless device, such as a hand-held wireless device (e.g., PDA), and all processing and database access occurs at the terminal 2.

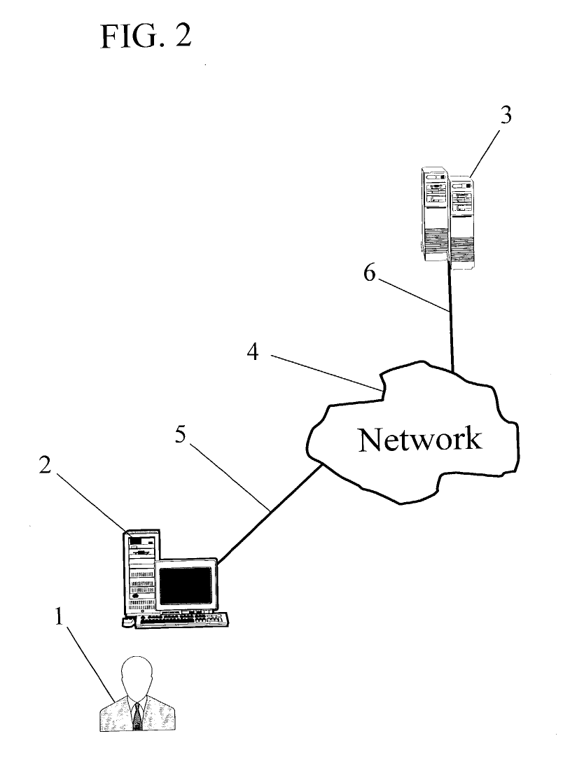

In a second embodiment, as shown in FIG. 2, data for use in the system is collected from a user 1 via a terminal 2 coupled to a server 3, such as a PC, minicomputer, mainframe computer, microcomputer, telephonic device, wireless device, or other device on a network 4, such as the Internet or an intranet. The terminal 2 can, for example, have or be accessible by a processor and/or have or be coupled to a repository for data via the network 4, and couplings 5, 6. The couplings 5, 6 include, for example, wired, wireless, or fiberoptic links.

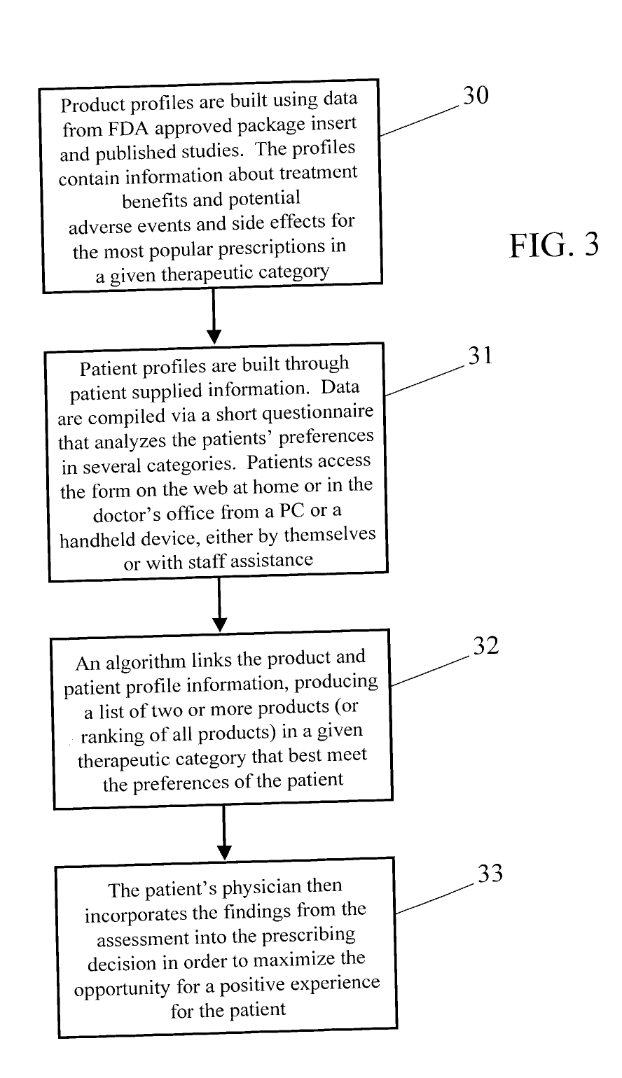

FIG. 3 is a flow chart of an overview of the data gathering an analysis method for individual patient medication preference applications, in accordance with an embodiment of the present invention. As shown in FIG. 3, product profiles are built using data from such sources as FDA approved package inserts and published studies 30. The profiles contain, for example, information about treatment benefits and potential adverse events and side effects for prescription medications applicable to each included therapeutic category, or for products under development.

Patient profiles are built through use of patient supplied information or otherwise supplied patient-specific information 31. Data are compiled via such mechanisms known in the art as a short questionnaire that provides information for use in analyzing the patients' preferences in several categories. The form is optionally accessible for the patients via, for example, a network, such as the Internet, using home computers, or via other devices known in the art, such as handheld devices. In another embodiment, each patient or the doctor, for example, is able to access the form at the doctor's office. Optionally, staff assistance is used to help the patient input information.

The product and patient profile information is used in the analysis and comparison process to produce a list of two or more products (or, for example, a ranking of all products for any number of products) in each selected therapeutic category, so as to best meet the preferences of the patient for that category or to otherwise allow use of such list or ranking information 32.

In one embodiment, the patient's health care provider (e.g., doctor) then incorporates the findings from the assessment into the prescribing decision in order to maximize the opportunity for a positive experience for the patient 33. The data is also useful for other purposes, such as for managed health care analysis of patient satisfaction and decisionmaking, or for marketing, research, and development assistant for drug manufacturers.

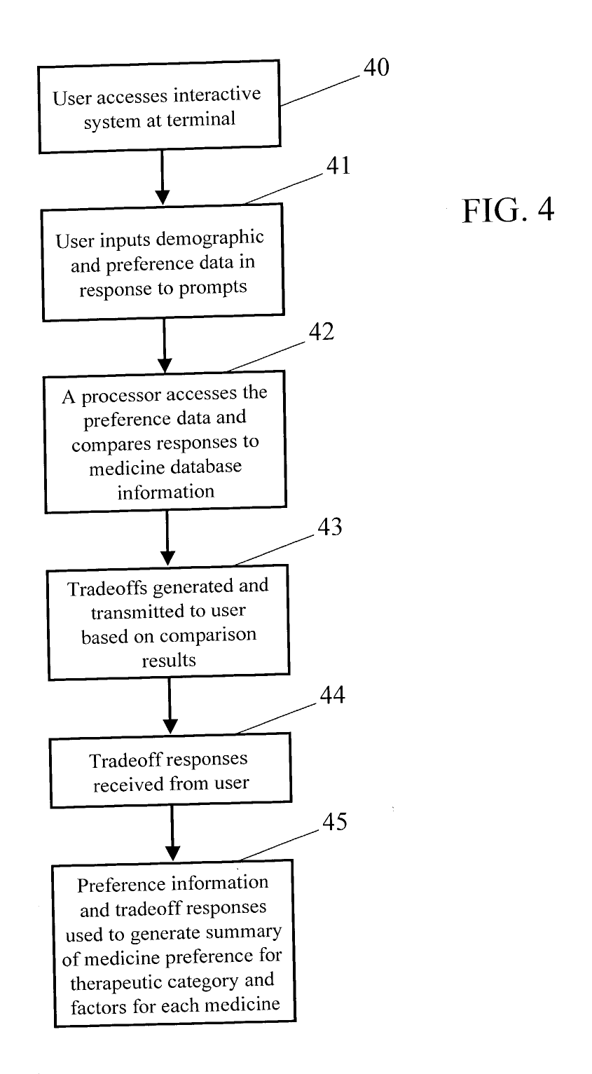

FIG. 4 is a flow chart of functions involved in an exemplary method for receiving and evaluating patient preference information, in accordance with an embodiment of the present invention.

As shown in FIG. 4, a user, such as a patient, a doctor, or a managed care provider accesses the interactive portion of the system using a terminal 40. The user inputs data 41, such as demographic and preference information. In one embodiment the interactive portion includes a series of prompts for information from the user. A processor, such as a server coupled to the terminal via a network, accesses the preference data and analyzes this data in conjunction with information contained in one or more other repositories, such as a database of therapeutic information and/or a database of cost information 42. As necessary, the processor then generates tradeoffs based on the compared and analyzed information and transmits the information to the user 43. The user then provides tradeoff responses 44.

The processor uses the preference information and tradeoff responses to generate a summary of treatment preference information, such as medicine preference by therapeutic category, along with factors or other information relating to the summary results 45. The user preferences or other results are then optionally provided to the user or, for example, to a treating physician.

Data and results may be presented in many formats, such as in reports customized to the user, to industry, or to other audiences. The data, results, and produced reports thus are able to serve as tools for driving market share (e.g., identifying products of potential high demand based on consumer preference), making clinical decisions, and for generating patient level market research data. In an embodiment of the present invention, output results may be customized, such as by preparing monthly reports targeted to specific purchasers of information and products subject to patient preference analysis.

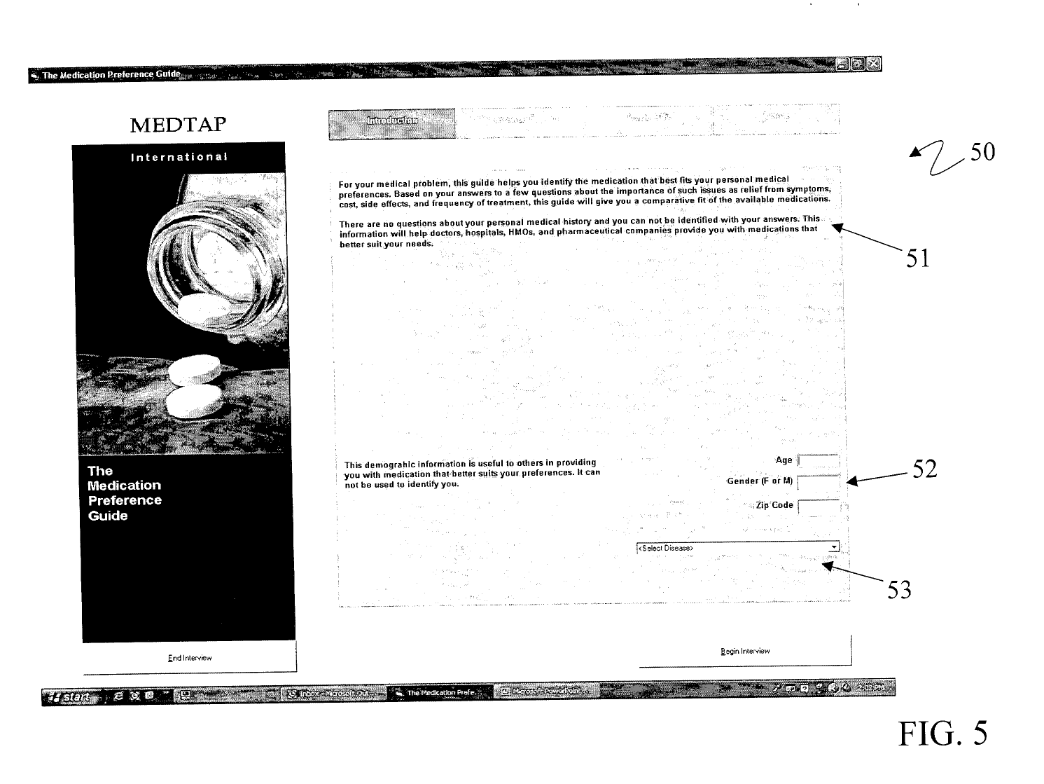

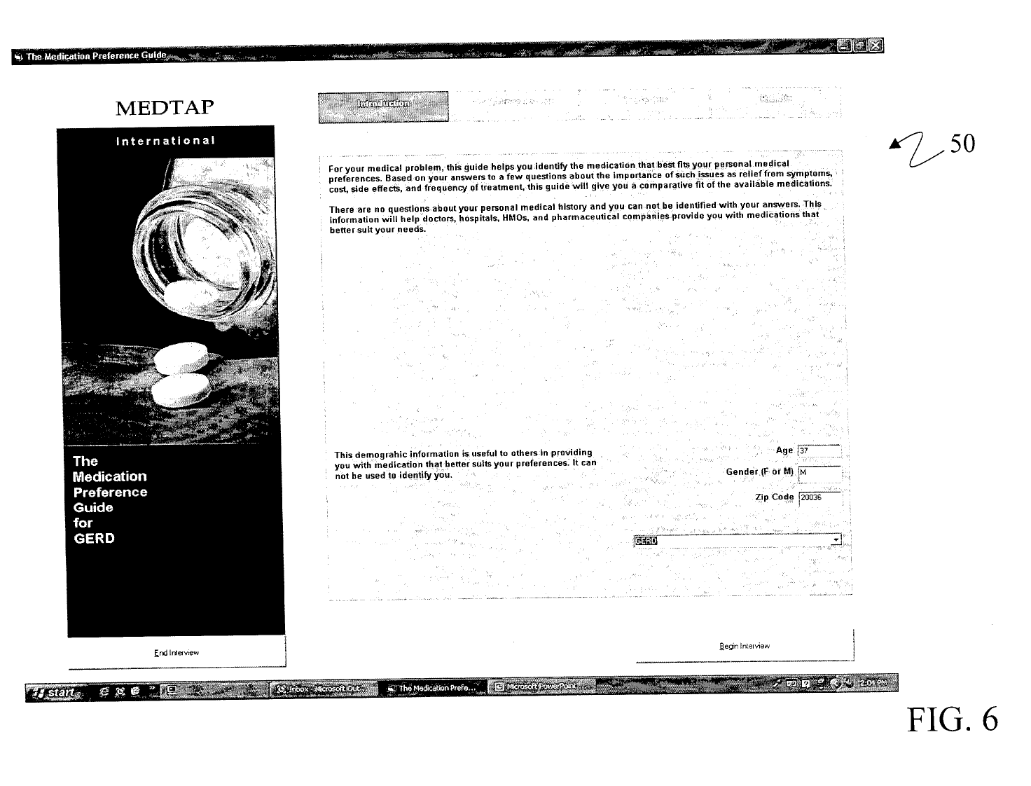

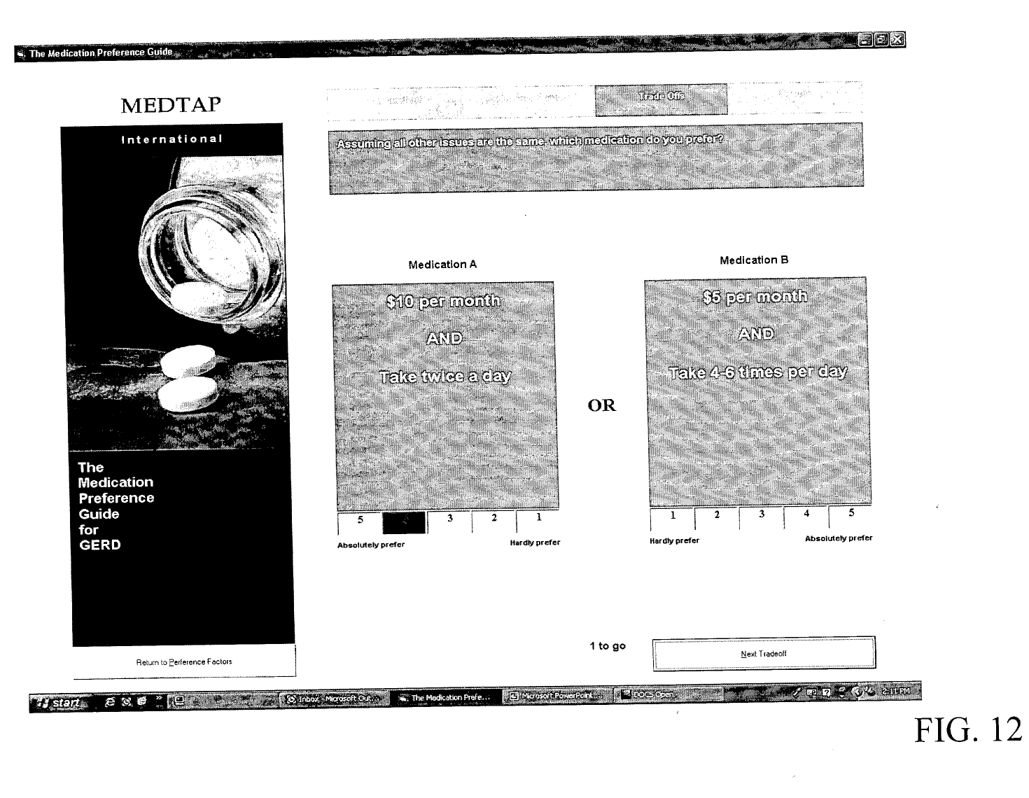

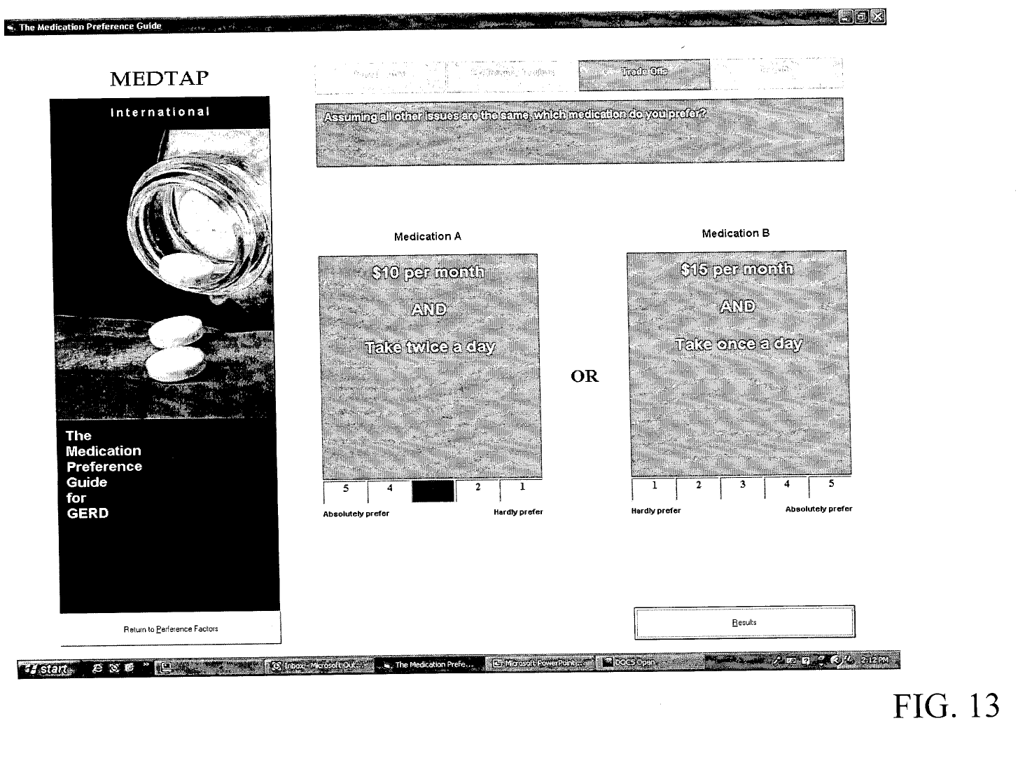

FIGS. 5-14 present sample graphical user interface (GUI) screens for patient preference data input, in accordance with an embodiment of the present invention. FIG. 5 shows an example introductory screen 50 that provides general explanatory information 51 and queries the user for demographic information 52, and prompts for a selection of a therapeutic category of interest 53. FIG. 6 presents the example screen 50 of FIG. 5 with sample input information.

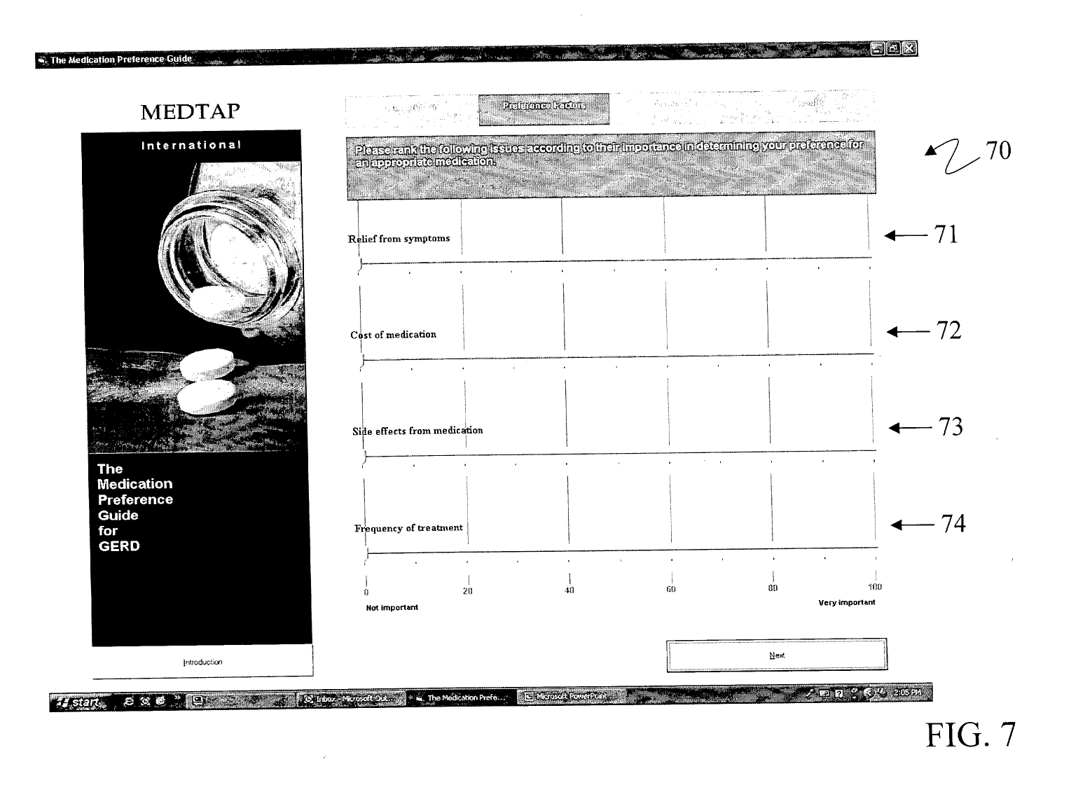

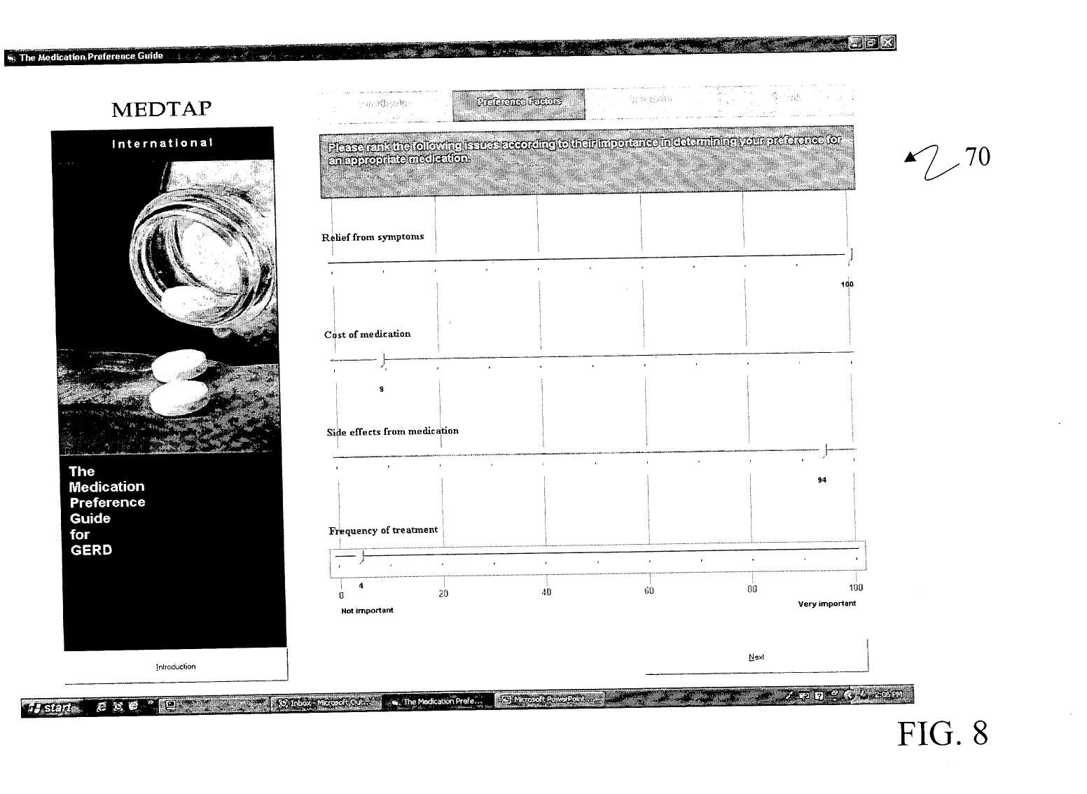

FIG. 7 contains an example preferences factors GUI screen 70, which allows user selected weighting of importance of various Preference Factors, such as relief from symptoms 71, cost of medication 72, side effect from medication 73, and frequency of treatment required by medication 74. Other factors that may be considered include, for example, mode of administration. FIG. 8 shows the screen 70 of FIG. 7 with sample input selections shown.

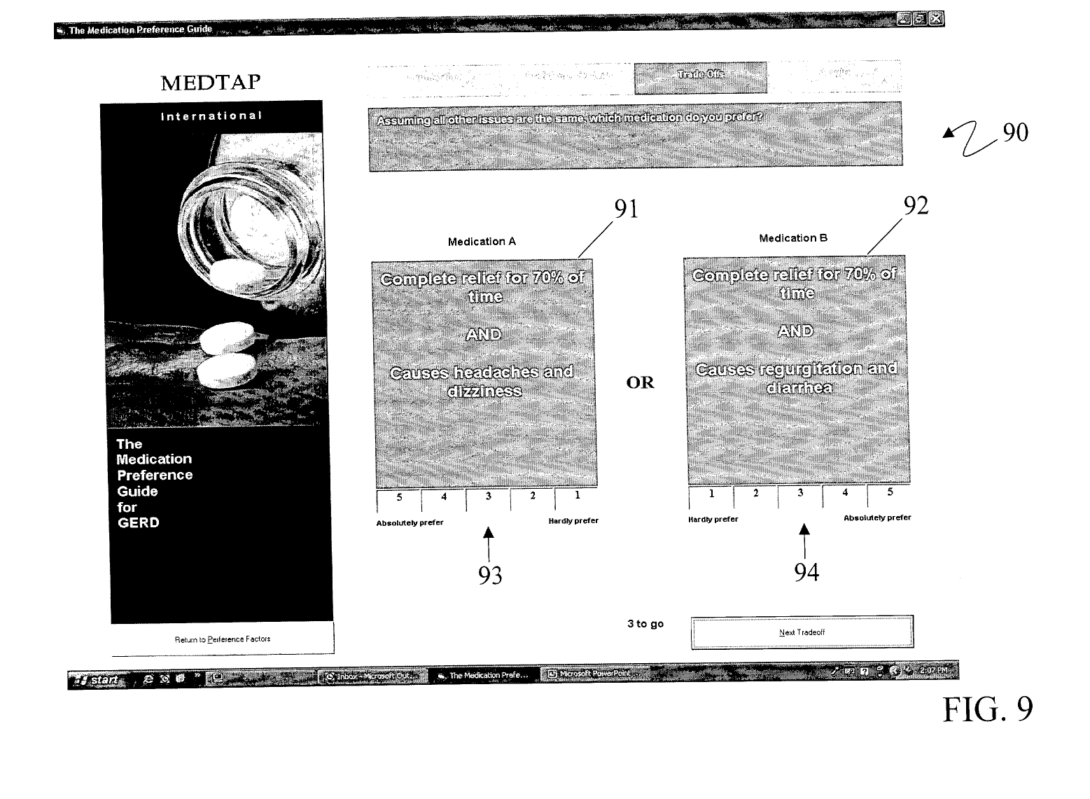

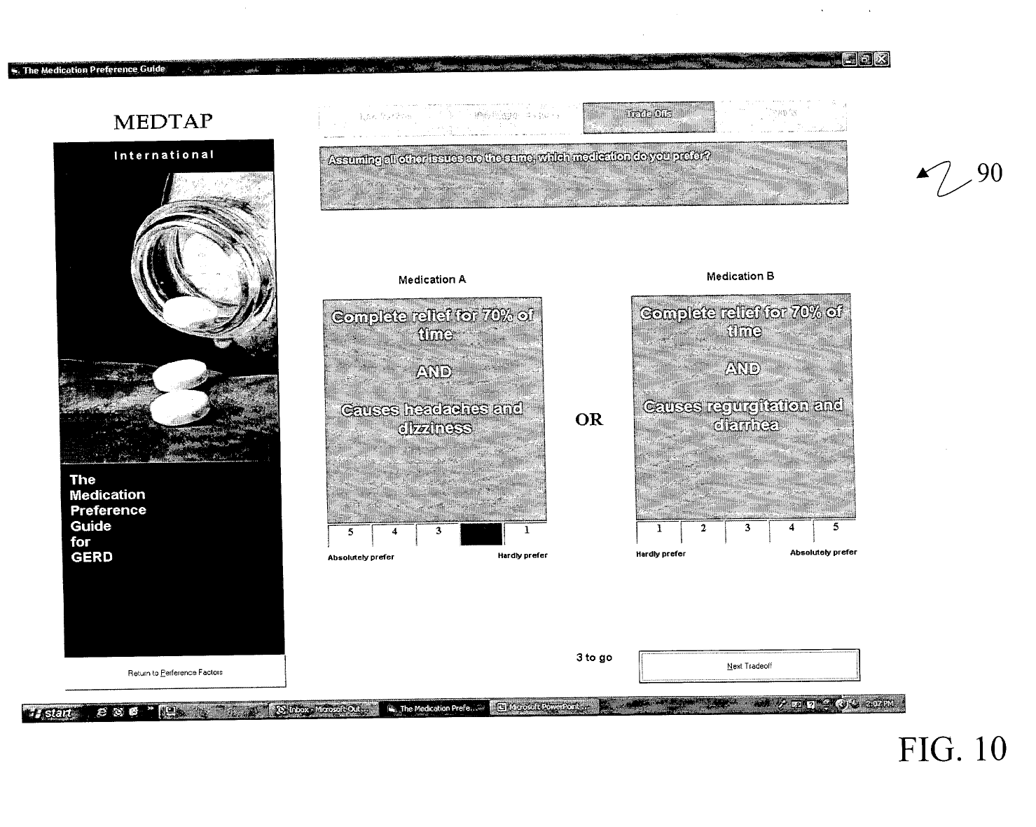

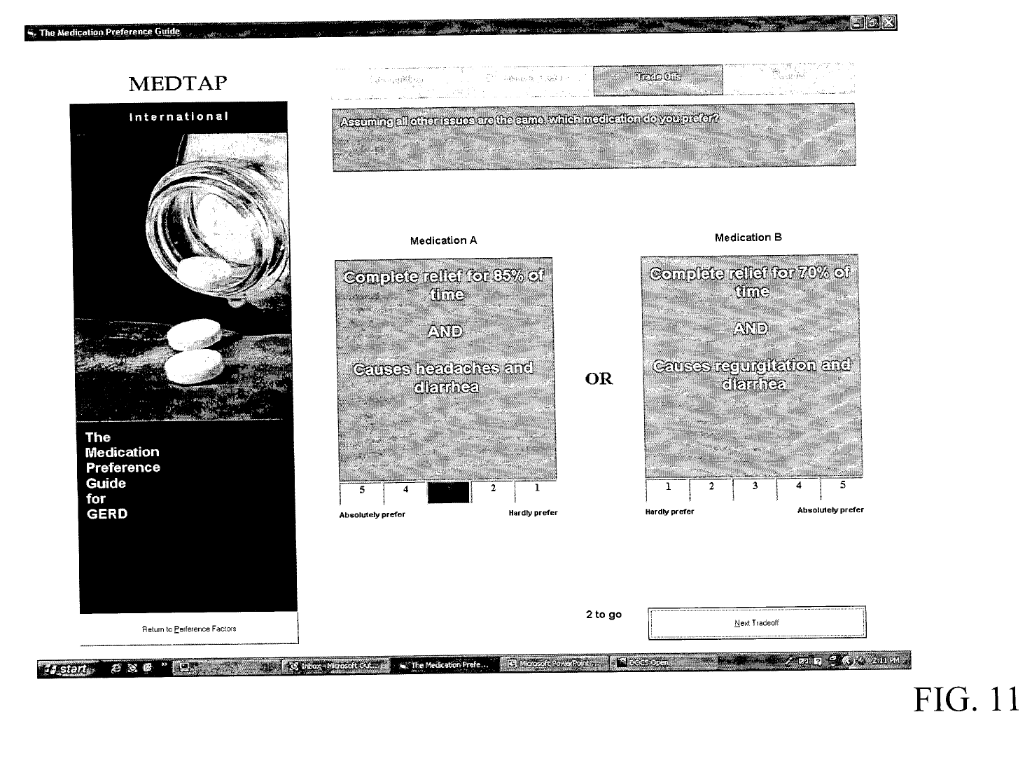

FIG. 9 is a screen 90 containing a first pair 91, 92 of example tradeoffs for two medications. The tradeoff pair includes user selectable preference scales 93, 94. FIG. 10 shows the screen 90 of FIG. 9 with a preference on the scale 93 selected. FIGS. 11-13 present additional tradeoff pairs of medications with preferences selected. One embodiment allows the selection of a preference applicable to only one member of each pair.

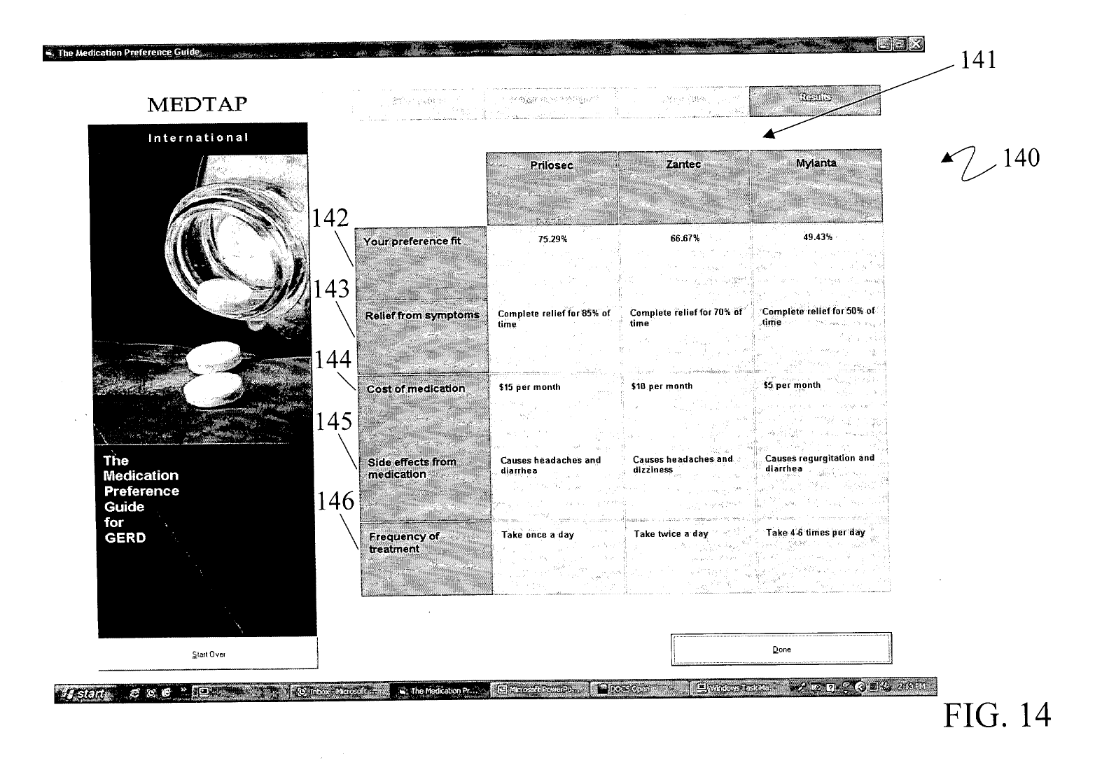

FIG. 14 shows a screen 140 with summary result information shown following application of the analysis and comparison process for three medications. As shown in FIG. 14, information presented includes columns of three medications at issue 141 and information on the user's preference fit 142, relief from symptoms 143, cost of medication 144, side effects from medication 145, and frequency of treatment required 146. Other information presented could include, for example, mode of administration.

Example embodiments of the present invention have now been described in accordance with the above advantages. It will be appreciated that these examples are merely illustrative of the invention. Many variations and modifications will be apparent to those skilled in the art.

What is claimed is:

1. A method for determining patient treatment information, comprising:
receiving treatment preference information from a user;
analyzing the received treatment preference information; and
evaluating the received treatment preference information using a repository of treatment option information to determine at least one preferred treatment option for the user.

2. The method of claim 1, further comprising:
receiving demographic information from the user.

3. The method of claim 1, wherein the treatment preference information includes medication treatment information.

4. The method of claim 3, wherein the medication treatment information includes information selected from a group consisting of relief from symptoms, cost, side effects, frequency of treatment, and mode of administration.

5. The method of claim 4, wherein the cost includes medication price.

6. The method of claim 4, wherein the cost includes user out-of-pocket expense.

7. The method of claim 1, wherein receiving treatment preference information from a user includes:
receiving scaled importance information from the user.

8. The method of claim 1, wherein receiving treatment preference information from a user includes:
receiving ranking information from the user.

9. The method of claim 8, further comprising:
producing at least one comparison query for two treatment options; and
receiving a user response to each of the at least one comparison query.

10. The method of claim 1, wherein analyzing the received treatment preference information includes:
weighting the received treatment preference information.

11. The method of claim 9, wherein analyzing the received treatment preference information includes:
weighting the received treatment preference information and each user response to each of the at least one comparison query.

12. The method of claim 1, wherein the repository of treatment option information includes medication specific information.

13. The method of claim 12, wherein the medication specific information is for a medication under development.

14. The method of claim 10, wherein the repository of treatment option information includes medication specific information.

15. The method of claim 14, wherein the medication specific information includes information selected from a group consisting of relief from symptoms, cost, side effects, frequency of treatment, and mode of administration.

16. The method of claim 14, wherein evaluating the received treatment preference information using a repository of treatment option information to determine at least one preferred treatment option for the user includes:
identifying a treatment condition; and
identifying a plurality of medications applicable to the treatment condition.

17. The method of claim 16, wherein evaluating the received treatment preference information using a repository of treatment option information to determine at least one preferred treatment option for the user further includes:
ranking each of the plurality of medications applicable to the treatment condition.

18. The method of claim 17, wherein ranking includes:
producing a weighted comparison of the received treatment preference information and the medication specific information for each of the plurality of medications applicable to the treatment condition.

19. The method of claim 1, further comprising:
evaluating the received treatment preference information using a repository of medication cost information to determine at least one preferred treatment option for the user.

20. The method of claim 19, wherein evaluating the received treatment preference information using a repository of medication cost information to determine at least one preferred treatment option for the user includes:
identifying medication cost information received from at least one managed health care organization.

21. The method of claim 20, wherein evaluating the received treatment preference information using a repository of medication cost information to determine at least one preferred treatment option for the user further comprises:
receiving identification of a user managed health care organization, the user managed health care organization being selected from the at least one managed care organization; and
identifying user specific medication cost information for the user managed health care organization.

22. The method of claim 1, wherein evaluating the received treatment preference information using a repository of treatment option information to determine at least one preferred treatment option for the user includes:
performing adaptive conjoint analysis.

23. The method of claim 1, wherein evaluating the received treatment preference information using a repository of treatment option information to determine at least one preferred treatment option for the user includes:
performing discrete choice model analysis.

24. The method of claim 1, further comprising:
providing the at least one preferred treatment option to a doctor.

25. The method of claim 1, further comprising:
providing the at least one preferred treatment option to the user.

26. The method of claim 1, further comprising:
collecting the received treatment preference information from the user in a repository of treatment preference information.

27. The method of claim 26, further comprising:
collecting the at least one preferred treatment option in the repository of treatment preference information.

28. The method of claim 27, further comprising:
receiving demographic information from the user; and
correlating the at least one preferred treatment option to the demographic information received from the user.

29. The method of claim 1, wherein the treatment preference information is received via a terminal.

30. The method of claim 29, wherein the terminal is selected from a group consisting of a minicomputer, a microcomputer, a mainframe computer, a telephone, and a hand-held device.

31. The method of claim 29, wherein the terminal is coupled to a network.

32. The method of claim 31, wherein the network is the Internet.

33. A method for determining medication preference information for a patient, the method comprising:
receiving a selection of a treatment of interest, the treatment of interest having a plurality of associated medications;
receiving the demographic information;
receiving a plurality of preference factor selections from the patient, the plurality of preference factor selections including at least one selected from a group consisting of valuation of relief from symptoms, cost of medication, side effects of medication, frequency of treatment, and mode of administration;
analyzing the plurality of associated medications with respect to the plurality of preference factor selections, wherein analyzing includes comparing pairs of the received plurality of preference factor selections and assigning a score for each of the plurality of preference factor selections based on a predetermined comparison threshold;
determining at least one tradeoff option for the plurality of associated medications, each of the at least one tradeoff option being identified from a repository of option information;
receiving a tradeoff selection for each of the at least one tradeoff option; and
scoring each of the plurality of associated medications, wherein scoring includes applying the score for each of the plurality of preference factor selections and a score applied for each of the received tradeoff selections;
wherein ranking includes weighting each of the plurality of associated medications based on the received preference factors information and the received tradeoff selection for each of the at least one tradeoff option.

34. A method for determining patient-specific treatment information for a patient, the method comprising:
receiving treatment preference information from the patient;
analyzing the received treatment preference information;
evaluating the received treatment preference information using a repository of treatment option information to determine at least one preferred treatment option for the patient; and
selecting a treatment option for the patient based on the at least one preferred treatment option.

35. A method for evaluating product preferences for a market sector, the method comprising:
receiving demographic information from a plurality of individuals in the market sector;
receiving treatment preference information from the plurality of individuals;
analyzing the received treatment preference information;
evaluating the received treatment preference information using a repository of treatment option information to determine at least one preferred treatment option for each of the plurality of individuals; and
determining an overall preference ranking for each of the at least one preferred treatment option for the plurality of individuals.

36. A system for determining patient treatment information, comprising:
a terminal having a processor;
a repository of treatment option information accessible via the terminal;
a repository of medication cost information accessible via the terminal; and
a repository of treatment preference information database accessible via the terminal;
wherein treatment preference information is collected in the repository of treatment preference information; and
wherein the processor evaluates the received treatment preference information using the repository of treatment option information and the repository of medication cost information to determine at least one preferred treatment option for the user.

37. The system of claim 36, wherein the terminal is selected from a group consisting of a minicomputer, a microcomputer, a mainframe computer, a telephone, and a hand-held device.

38. The system of claim 36, wherein the terminal is coupled to a network.

39. The system of claim 38, wherein the network is the Internet.

40. The system of claim 38, wherein the repository of treatment option information is contained in a server on the network.

41. The system of claim 38, wherein the repository of medication cost information is contained in a server on the network.

42. The system of claim 38, wherein the repository of treatment preference information is contained in a server on the network.

43. A system for determining patient treatment information, comprising:
means for receiving treatment preference information from a user;
means for analyzing the received treatment preference information; and
means for evaluating the received treatment preference information using a repository of treatment option information to determine at least one preferred treatment option for the user.

44. A computer program product comprising a computer usable medium having control logic stored therein for causing a computer to determine patient treatment information, the control logic comprising:
first computer readable program code means for causing the computer to receive treatment preference information from a user;
second computer readable program code means for causing the computer to analyze the received treatment preference information; and
third computer readable program code means for causing the computer to evaluate the received treatment preference information using a repository of treatment option information to determine at least one preferred treatment option.
